(19) United States
(12) Patent Application Publication
(10) Pub. No.: US 2010/0017894 A1
(43) Pub. Date: Jan. 21, 2010

(54) Mutants of Human App and Their Use for the Production of Transgenice Animals

(76) Inventors: Dirk Beher, Vaud (CH); Jonathan David Wrigley, Cheshire (GB)

Correspondence Address: MERCK AND CO., INC, P O BOX 2000, RAHWAY, NJ, 07065-0907, US

(21) Appl. No.: 11/922,050

(22) PCT Filed: Jun. 13, 2006
(86) PCT No.: PCT/GB2006/002157

§ 371 Date: Oct. 6, 2009

(30) Foreign Application Priority Data
Jun. 13, 2005 (GB) 0511861.7

(51) Int. Cl. G01N 33/00; C12N 15/63; C12N 5/10; A01K 67/00; C12Q 1/02

(52) U.S. Cl. 800/3; 435/320.1; 435/325; 800/13; 435/29

(57) ABSTRACT

A modified amyloid precursor protein can be expressed from a DNA construct comprising an APP DNA sequence, or a fragment or artificial substrate thereof, encoding the mutation I45W, I45Y V46W or V46Y (numbering relative to A4CT); the use of the DNA construct in the generation of cell lines or transgenic animals and the use of these proteins or such transgenic animals in the diagnosis of Alzheimer's Disease and screening putative drugs against Alzheimer's Disease are also described.

The present invention relates to modified amyloid precursor proteins, their use in the generation of cell lines, their use in the generation of transgenic animals and the use of these proteins or such transgenic animals in the diagnosis of Alzheimer's Disease and screening putative drugs against Alzheimer's Disease.

One of the main hallmarks of Alzheimer's disease (AD) is the progressive formation of senile plaques in the brain of affected individuals. These extracellular deposits are comprised of amyloid-β (Aβ) peptides (Glenner O. G. et al. (1984) Biochem Biophys Res Commun 120, 885-890; Masters C. L. et al. (1985) Proc Natl Acad Sci USA 82, 4245-4249) which according to the “amyloid cascade hypothesis” (Hardy J. A. et al. (1992) Science 256, 184-185) are the causative agents for this debilitating condition.

γ-Secretase is a critical enzyme in the pathogenesis of Alzheimer's Disease, being responsible for the final cleavage of βAPP (amyloid precursor protein) to release the Aβ peptides. The exact mechanism of cleavage, like the structure of the enzyme, remains unknown, although there is believed to be a loose sequence specificity since a wide range of Aβ peptides of varying length are produced. Whilst the majority of Aβ peptides exist as a 40 amino acid species, it is the production of a more aggregatory 42 amino acid product that is believed to lie at the heart of disease progression, since this is a common feature of all mutations that are causative for early-onset, familial AD (FAD).

Genetic studies have identified numerous mutations within both the catalytic site of the enzyme, presenilins PS1, and the substrate, βAPP, which are causative for early-onset, familial AD (FAD) (Bertain L. et al. (2004) Pharmacol Res 50, 385-396). The common feature of these mutations is that they result in enhanced generation of the highly pathogenic Aβ(42) peptide.

Patent application WO 98/58060 describes constructs comprising a human APP or A4CT DNA sequence encoding mutations which lead to a higher ratio of Aβ4(1-42)/Aβ4(1-40) than wild type and their use in the production of transgenic animals developing amyloid plaques as a model of Alzheimer's Disease. Constructs encoding the mutation I45F (numbering relative to A4CT) and optionally additionally coding a mutation selected from V46F, V46I, V46G, V46Y, V46L, V46A, V46P, V46W, V46M, V46S, V46T, V46N or V46Q are disclosed although only constructs encoding I45F, and I45F together with V46F are exemplified. Transgenic rodents containing such constructs are also described as is their use in drug screening.

The construction of transgenic mammals expressing βAPP is also disclosed, for example, in US patent application 2002/0104104A.

The present invention relates to the identification of four novel mutations that result in enhanced production of the Aβ(42) peptide. These mutations all reside around the γ-secretase cleavage site within βAPP, and result in the equivalent or larger enhancements of Aβ(42) than any previously reported mutations within βAPP.

Furthermore, some of these mutations demonstrate a concomitant reduction in Aβ(40) production and a common alteration in the ratios of different Aβ peptides generated from these mutated substrates.

Thus, in a first aspect, the present invention provides a DNA construct comprising an APP DNA sequence, or a fragment or artificial substrate thereof, encoding the mutation I45W, I45Y, V46W or V46Y (numbering relative to A4CT). Suitably the DNA sequence is a human DNA sequence and reference to human APP includes all isoforms, including the 695 form. Additionally, the references herein to A4CT, Aβ40 and Aβ42 include all N-terminal variants produced by alternative cleavage during processing. A suitable fragment of APP is an A4CT DNA sequence. The construct may contain combinations of the identified mutations at positions 45 and 46 as well as single mutations.

In one embodiment, the construct comprises an A4CT DNA sequence, for example an SPA4CT sequence. The product of this cDNA contains the signal peptide of βAPP spliced to a leucine, a glutamic acid, and then the carboxy-terminal 99 amino acids of βAPP. This sequence of βAPP corresponds to the β-CTF generated by β-secretase cleavage, and this substrate therefore only requires cleavage by γ-secretase to liberate Aβ peptides.

The present invention also provides mammalian cells expressing the construct and vectors containing it.

Mammalian cells expressing the construct may be prepared by conventional methods.

Host cells are genetically engineered (transduced or transformed or transfected) with the vectors of this invention which may be, for example, a cloning vector or an expression vector. The vector may be, for example, in the form of a plasmid, a viral particle or a phage. The engineered host cells can be cultured in conventional nutrient media modified as appropriate for activating promoters, selecting transformants or amplifying the human genes. The culture conditions, such as temperature, pH and the like will be apparent to the ordinary skilled artisan.

Various mammalian cell culture systems can be employed to express recombinant protein. Examples of mammalian expression systems include the HEK293 lines of human embryonic kidney cells, and other cell lines capable of expressing a compatible vector, for example, the SH-SY5Y, CHO, COS-7 and HeLa cell lines.

The selection of an appropriate host is deemed to be within the scope of those skilled in the art from the teachings herein.

Mammalian expression vectors will comprise an origin of replication, a suitable promoter and enhancer, and also any necessary ribosome binding sites, polyadenylation sites, splice donor and acceptor sites, transcriptional termination sequences, and 5′ flanking non transcribed sequences. DNA sequences derived from the SV40 splice, and polyadenylation sites may be used to provide the required nontranscribed genetic elements.

In addition, the expression vectors preferably contain one or more selectable marker genes to provide a phenotypic trait for selection of transformed host cells such as hygromcyin or neomycin resistance for eukaryotic cell culture.

The appropriate DNA sequence may be inserted into the vector by a variety of procedures. In general, the DNA sequence is inserted into an appropriate restriction endonuclease site(s) by procedures known in the art. Such procedures and others are deemed to be within the scope of those skilled in the art.

The DNA sequence in the expression vector is operatively linked to an appropriate expression control sequence(s) (promoter) to direct mRNA synthesis. Examples of such promoters include the CMV promoter, pCEP4 (Invitrogen) and other promoters known to control expression of genes in eukaryotic cells or their viruses and replicable and viable in the host.

Introduction of the construct into the host cell can be effected by calcium phosphate transfection, lipofectin-mediated transfection, or electroporation. (Davis, L., Dibner and M., Battey, I., Basic Methods in Molecular Biology, (1986)).

In a further aspect, the present invention provides a non-human transgenic mammal whose cells contain a construct as hereinbefore described.

The transgenic mammal is produced by conventional techniques (Hogan et al., ‘Manipulating the mouse embryo’, Cold Spring Harbor Laboratory, Cold Spring Harbor (1994); C. A. Pinkert ed. ‘Transgenic animal technology, a Laboratory Handbook (Academic Press Inc. 1994); D. Murphy and D. A. Carter ed. ‘Transgenesis Techniques’ Principles and Protocols (Humana Press Totowa, N.J. 1993); E. J. Robertson ed. ‘Teratocarcinomas and Embryonic Stem Cells’, a Practical Approach (IRL Press 1987)).

In one aspect, the transgenic mammal is produced by introduction of the construct into an embryo, insertion of the embryo into a surrogate mother and allowing the embryo to develop to term.

The construct is prepared for transfer to the host animal by cleavage of vector containing the construct and purification of the DNA (Hogan et al., ‘Manipulating the mouse embryo’, Cold Spring Harbor Laboratory, Cold Spring Harbor (1994)).

The transfer is carried out conventionally preferably using a microinjection as described in Hogan et al. above.

In an alternative aspect the transgenic mammal is produced by introduction of the construct into embryonic stem cells by conventional methods such as calcium phosphate/DNA precipitation, direct injection or electroporation (E. J. Robertson ed. ‘Teratocarcinomas and Embryonic Stem Cells’, a Practical Approach (IRL Press 1987)) followed by injection of the transformed cells into blastocytes and insertion of the resulting embryo into a surrogate mother as described above.

Transgenic animals are identified by DNA analysis using Southern blot and PCR to detect founder animals.

The transgenic mammal is preferably a rodent such as rat or mouse, more preferably a mouse.

The APP or A4CT coding DNA is obtained by probing a human cDNA library. Mutations may be introduced by site-directed mutagenesis or during construction of the coding DNA from appropriate fragments.

Suitable promoters for use in the present invention include: Human APP (Wirak D. O., et al., (1991) Science, 253, 323-325); rat neuron specific enolase (neurons) (Forss-Petter et al., (1990) Neuron, 5, 197); human β actin (Ray et al., (1991) Genes and Development, 5, 2265-2273); human PDGFβ (Sasahara et al., (1991) Cell, 64, 217-227); mouse Thy 1 (Ingraham et al., (1986) Mol. Cell. Biol., 6(8), 2923-31); mouse Prion protein promoter (PrP) (Fischer et al., (1996) EMBO J., 15(6), 1255-64); Syrian hamster Prion protein promoter (Scott et al., (1992) Prot. Sci., 1, 986-997); rat synapsin 1 (brain) (Howland et al., (1995) Neurobiol. Aging, 16(4), 685-99); human FMR1 (brain) (Hergersberg et al., (1995) Hum. Mol. Genet., 4(3), 359-66); human neurofilament low (Thomas et al, (1994) J. Virol, 68(11), 7099-107), middle (brain) (Tu et al., (1995) J. Cell. Biol., 129(6), 1629-40); NEX-1 (brain) (Bartholoma et al., (1994) Mech. Dev., 48(3), 217-8); mouse APLP2 (brain) (Kock et al., (1995) J. Biol Chem., 270(43) 25475-80); rat alpha tubulin (Gloster et al., (1994) J. Neurosci., 14(12), 7319-30); mouse transferrin (Thiesen et al., (1993) Mol. Cell. Biol., 13(12), 7666-76); mouse HMGCR (3-hydroxy-3-methylglutaryl coenzyme A reductase, oligodendrocytes) (Duhamel-Clerin et al., (1994) Glia, 11(1), 35-46) and mouse myelin basic protein (Readhead et al., (1987) Cell, 48, 703-712).

A tetracycline-inducible system may also be used, which has the advantage of regulating the gene expression (induction/repression) (Furth et al., (1994) PNAS USA, 91, 9302-9306; Baron et al, (1995) Nucl. Acids Res., 23(17), 3605-6). This system uses two constructs: a minimal promoter (PhCMV*-1) fused to seven tetracyclic operator sequences and the cDNA in question; and a transgene containing the tetracycline-controlled transactivator protein (tTA) coding sequence under the control of a promoter, for example taken from the above list. Each construct is used to generate a transgenic mouse. Crossing the two transgenic mice can generate a double transgenic line which expresses the tTA according to the chosen promoter. This tTA induces expression of the cDNA by activating the PhCMV*-1, but only in the absence of a tetracycline. In the presence of tetracycline there is only basal expression.

A preferred promoter is the human PDGFβ (Sasahara et al., (1991) Cell, 64, 217-227), mouse Prion protein promoter (Fischer M. et al., (1996) EMBO J., 15(6), 1255-64) or hamster Prion protein promoter (Scott et al., (1992) Prot. Sci., 1, 986-997). A preferred splice form of APP is APP695, which is the major splice form expressed in the mammalian brain, but APP751 or APP770 can also be used. The construct is prepared by conventional recombinant DNA techniques (Maniatis et al., ‘Molecular Cloning, a Laboratory Manual’, Cold Spring Harbor Laboratory, Cold Spring Harbor (1989)) and the codon change, for example V46W, introduced into the human APP clone, for example the APP695cDNA clone, by conventional site-directed mutagenesis. The mutation can be labeled with novel restriction sites such as AlwI, BsaBI, MamI and Sau3AI sites, which allows identification in the transgenic animal. The construct can also include SV40 intron/poly A sequences to stabilize the mRNA. The transgenic construct may contain either the single mutation, for example APP695V46W, or a double mutation, the latter of which would further boost Aβ1-42 production by having the γ-secretase enhancing NFEV mutation, for example APP695NFEV and V46W. The single or double mutant construct can be introduced by the methods listed above; the preferred method being pronuclear injection into either mouse (e.g. C57BL6/J) or rat (e.g. Long-Evans or Spraque-Dawley). Transgenic founder animals are test bred for germ line transmission with the respective wild-type rodent. Expression levels of the transgene will depend on copy numbers and integration site of the transgene. The most successful transgenic mouse models all have high copy numbers and high expression levels (Price D. L. et al., (1998) Annu. Rev. Genet. 32, 461-93). Aβ1-42 levels and human APP expression can be tested in the transgenic rodents and the highest expressing line further propagated for the studies.

In another aspect, the present invention provides a method for screening putative drugs by administering the test drug to the transgenic mammal or cell culture medium hereinbefore described and observing changes in APP expression and processing, histopathology and/or behavioural changes. Preferably, such histological and/or behavioural changes are associated with Alzheimer's Disease.

Suitable techniques for making such observations are well known to those skilled in the art, for example quantifying amyloid plaques by uniform random sampling of serially sectioned caudal quadrants. Electrophysiological changes can be measured for example by Long-term potentiation in hippocampal slices, which has been postulated to be an underlying mechanism of learning and memory. Reversal of behavioural impairments by treatment with suitable Aβ lowering drugs can be demonstrated in learning and memory assays such as the Morris watermaze task, T-maze alternation task, 5-Choice serial reaction time task (5-CSRT) or contextual renewal task (CR).

Such assays include the use of these mutations in cell-based assays, in vitro assays using membrane preparations from these cells, and assays using solubilised enzyme derived from these cells. The measurement of Aβ produced in these assays may be used to evaluate the effects of compounds and other mutations for example, as well as providing mechanistic data on γ-secretase and the βAPP processing pathway. Examples of the methods utilized for such studies have been described (Wrigley et al., (2004) J. Neurochem, 90, 1312-1320, and Wrigley et al., (2005) J. Biol. Chem., 280, 12523-12535).

The following examples illustrate the preparation of constructs of the present invention and the use of such constructs.

### EXAMPLES

Materials—βAPP and its C-terminal fragments were detected using affinity purified polyclonal rabbit antibody R7334 (Beher et al (2001) J Biol Chem 276, 45394-45402)(1:2,500 dilution). The secondary antibody used was HR-conjugated polyclonal goat anti-rabbit F(ab′)₂ fragments (Amersham, 1:5,000 dilution).

Sulindac sulfide and fenofibrate (Calbiochem and Sigma-Aldrich respectively) were prepared as 200 mM stocks in DMSO and diluted as indicated in the figures.

Complementary DNA constructs and site-directed mutagenesis—A series of mutations were introduced into the cDNA for the direct γ-secretase substrate SPA4CT (Dyrks T. et al. (1993) FEBS Lett 335, 89-93). (A construct containing the cDNA for the SPA4CT construct was obtained from Konrad Beyreuther). Site-directed mutagenesis was performed using the Quikchange™ mutagenesis kit (Stratagene) according to the manufacturer's instructions. Successful mutagenesis was confirmed by double-stranded automated DNA sequencing (CEQ-DTCS—Quick Start kit, Beckman).

Cell culture, transfections and cell lines—The wild-type and mutant SPA4CT cDNAs were transfected into wild-type HEK293 (human embryonic kidney 293) cell line propogated under standard conditions. One day post transfection the media on the cells was replaced and 24 hours later the conditioned media from these cell lines was removed for analysis. For transient transfection 2×10⁶ cells were plated into 10 cm diameter dishes 1 day prior to treatment. Transfections were performed with 10 μg of the appropriate SPA4CT construct (Dyrks T. et al. (1993) FEBS Lett 335, 89-93) using the Gene Juice transfection reagent (Novagen).

Quantification of Aβ peptides in conditioned cell media—Aβ peptides secreted into the media (from wild type and mutant SPA4CT cDNAs) were quantified using an ECL assay based on a previously described method (Lin 2000). Samples of 25 and 50 μl of media were used for detection of Aβ(40) and Aβ(42) respectively, the Aβ(40) samples being supplemented with 25 μl of assay buffer (PBS, 2% BSA, 0.2% Tween-20). The Aβ(40) and Aβ(42) samples were transferred to an Avidin-coated Meso-Scale Discovery plate and supplemented with 25 μl of assay buffer containing 0.16 μg/μl 4G8-Biotin and either 0.04 μg/μl Ruthenylated G2-10 or G2-11 respectively. Following overnight incubation at 4° C., 200 rpm, the media was discarded and the plate washed three times with PBS, prior to addition of 150 μl Meso-S Read Buffer and analysis using the Meso-Scale Discovery analyzer (Sector Imager 6000). The levels were then displayed relative to secretion from the cells transfected with wild type SPA4CT (FIG. 2A).

Membrane preparation and solubilisation of membrane proteins for Western blot analysis—The cells were lysed and membrane proteins solubilised as described ((Beher et al. (2001) J Biol Chem 276, 45394-45402). Protein concentrations of the resultant samples were determined using the bicinochoninic acid assay (Hill H. D. et al. (1988) Anal Biochem 170, 203-208) in a 96-well plate format, according to the manufacturer's instructions (Perbio). Equal amounts of protein were separated by sodium dodecyl sulphate polyacrylamide gel electrophoresis (SDS-PAGE) using 10-20% Tricine gels (Invitrogen). Proteins were transferred to nitrocellulose membranes, and probed with antibodies as indicated in the figures, using the enhanced chemiluminescence system (ECL, Amersham) (FIG. 2B).

Surface-enhanced Laser Desorption/Ionization Time-of-Flight (SELDI-TOF) Mass Spectrometry—Immunocapture of Aβ peptides from conditioned cell media was performed analogous to previously described methods using the monoclonal antibody 6E10 and purified non-immune mouse IgG as a negative control (Beher et al. (2002) J Neurochem 82, 563-575). Briefly, following collection the conditioned media was diluted to 0.5% (v/v) Triton X-100, 25 mM HEPES pH 7.3 (10 ml final volume). Following a centrifugation for 10 min at 4,000 g the supernatant was incubated overnight at 4° C. with the antibody-coupled SELDI protein chip (PS1 preactivated surface ProteinChip array; Ciphergen Biosystems, Fremont) which was processed for SELDI-TOF mass spectrometry as described previously.

Results

Novel Mutations Within βAPP with Aβ(42)-Elevating Properties

In order to determine the effect of these mutations on Aβ generation and levels of secreted Aβ(40) and Aβ(42) the mutant constructs were analysed as described in the previous section). These data demonstrate that as reported previously (Lichtenthaler et al. (1999) Proc Natl Acad Sci USA 96, 3053-3058 the I45F mutation results in an elevation in Aβ(42) and a concomitant reduction in Aβ(40) secretion. The novel I45W, I45Y, V46W and V46Y mutations also give large increases in secreted Aβ(42) levels, varying from two-fold to three-fold. Notably, unlike all of the other mutations analysed the V46W mutation gave only a slight reduction in Aβ(40) secretion, leaving levels of this peptide relatively unaffected.

SELDI-TOF Analysis of Secreted Peptides Demonstrates a Common Mechanism

In an attempt to characterize the mechanism by which these changes in Aβ peptide secretion occur, the range of Aβ peptides secreted into the media was analysed by SELDI-TOF mass spectroscopy. The data demonstrate that a whole range of peptides are generated by processing of the wild-type SPA4CT construct (see FIG. 3). Aβ(1-40) is the predominant product, however the Aβ(1-37), Aβ(1-38), Aβ(1-39) and Aβ(1-42) are also detectable. In contrast notable changes are observed in the Aβ peptides present in media from cells transfected with mutant SPA4CT (FIGS. 2B-F). In all cases Aβ(1-42) production is elevated, in agreement with the data obtained in the ECL assay. In addition, all of the mutations also result in elevated levels of both Aβ(1-38) and Aβ(1-39).

Furthermore, as demonstrated using the ECL assay, both the I45W and I45Y mutations reduce Aβ(40) levels to almost undetectable levels, whereas the V46W and V46Y mutations result in smaller reductions in Aβ(40) generation. However, the reduction of Aβ(40) does appear to represent a common mechanism.

### FIGURE LEGENDS

FIG. 1. Schematic representation of SPA4CT and engineered mutations

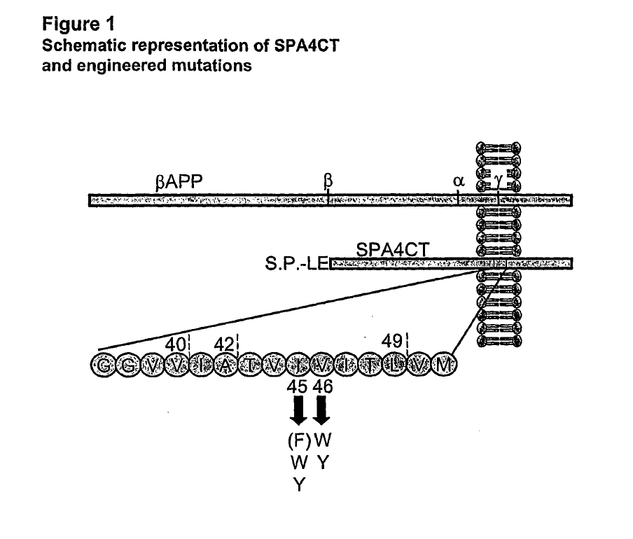

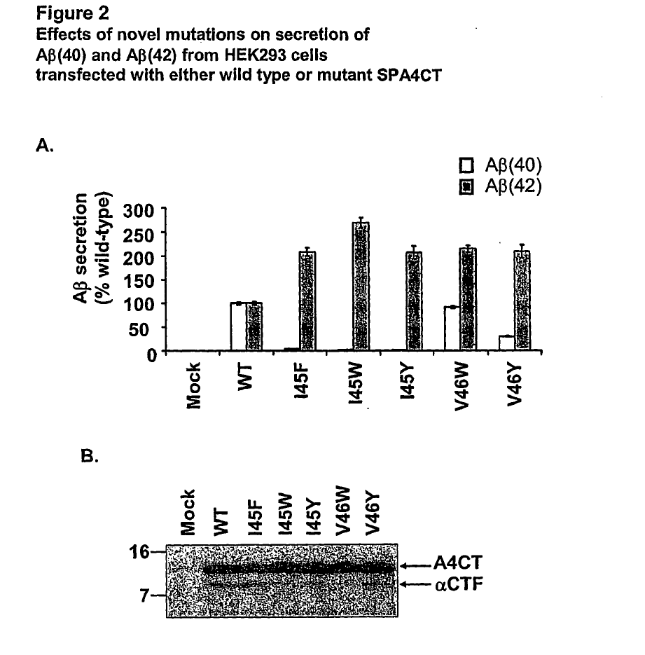

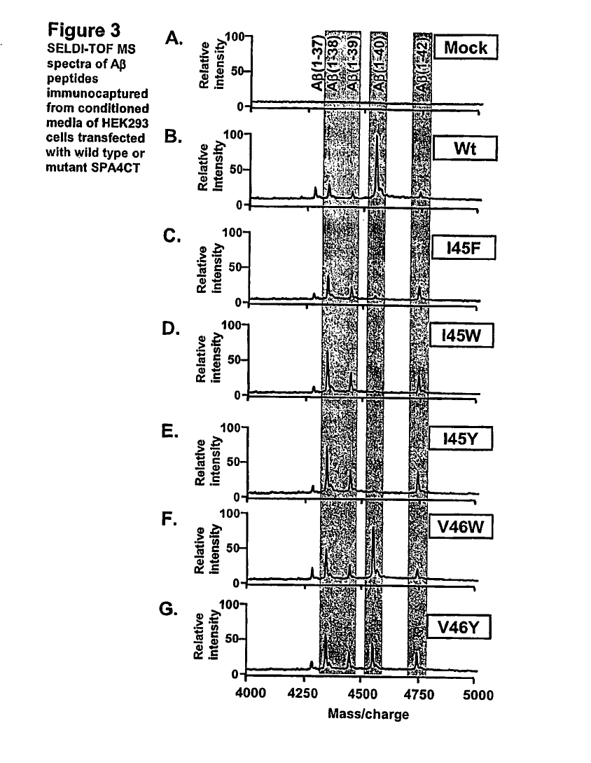

SPA4CT comprises the signal peptide of βAPP (S.P.) spliced to a leucine and a glutamic acid residue (LE) adjacent to the C-terminal 99 amino acids of βAPP. This correlates to the region of βAPP from the β-cleavage site to the C-terminal end. Due to the exclusion of the ectodomain from this product, SPA4CT acts as a direct substrate for γ-secretase cleavage with no prerequisite cleavages apart from the constitutive SPP cleavage upon biosynthesis. Numbering of the sequence of the SPA4CT protein and products thereof begins at the residue directly C-terminal of the LE sequence, corresponding to the first amino acid of the native Aβ peptide. Mutations were incorporated at positions 45 and 46, the original residues in the circles being replaced with the residues indicated below the arrows at each of these positions. The I45F mutation is in parentheses since this is a previously reported mutation, used for comparative purposes. The major cleavage sites, after residues 40, 42 and 49 are indicated by the dashed lines.

FIG. 2. Effects of novel mutations on secretion of Aβ(40) and Aβ(42) from HEK293 cells transfected with either wild type or mutant SPA4CT.

Wild type HEK293 cells were transfected with either wild type or mutant SPA4CT as indicated and conditioned media collected 24 hours post-transfection. (A) Secreted Aβ peptides were assayed using the Meso-Scale Discovery ECL assay, and secreted peptides are displayed as a percentage of peptide levels in media from cells transfected with the wild type SPA4CT plasmid. Error bars indicate the standard error of the mean of the combined values from the duplicate transfections with quadruplicate Aβ measurements for each. (B) Additionally, equal amounts of solubilised membrane protein (50 μg protein) from each dish were separated by SDS-PAGE and immunblotted for βAPP s (C-terminal fragments) using the polyclonal rabbit antiserum R7334 and horseradish peroxidase conjugated goat anti-rabbit secondary antibody. A representative Western blot obtained from one set of the duplicate transfections is shown.

FIG. 3. SELDI-TOF MS spectra of Aβ peptides immunocaptured from conditioned media of BEK293 cells transfected with wild type or mutant SPA4CT. Aβ peptides were immunocaptured from conditioned media using the monoclonal antibody 6E10. Captured peptides were directly analysed by SELDI-TOF MS. Spectra are displayed for conditioned media from cells transfected with empty vector (A), wild type SPA4CT (B), or SPA4CT bearing the I45F (C), I45W (I)), I45Y (E), V46W(F) or V46Y (Gy6) mutations. All spectra were normalized to the average intensity height of the peak for the single charged bovine insulin peptide species and calibrated internally using the single and double positively charged species of bovine insulin.

Note that due to the cloning of the original SPA4CT construct, all Aβ peptides derived from the SPA4CT protein contain the additional two amino acids leucine and glutamic acid at the N-terminus (Dyrks T. et al. (1993) FEBS Lett 335, 89-93), the last C-terminal amino acid indicating the last amino acid of the native Aβ sequence. The data are representative of a minimum of two independent experiments.

1. A DNA construct comprising DNA sequence encoding APP, or a fragment or artificial substrate thereof, and where said DNA sequence encodes the mutation I45W, I45Y, V46W or V46Y, wherein said mutation is numbered relative to the APP fragment, A4CT.

2. A construct of claim 1 wherein said APP fragment is A4CT.

3. A mammalian cells expressing a construct of claim 1.

4. A vectors comprising a construct of claim 1.

5. A non-human transgenic mammal whose cells contain a construct of claim 1.

6. A method for screening putative drugs by administering a test drug to a transgenic mammal of claim 5 and observing changes in APP expression and processing, histopathology or behavior.

7. A method for screening putative drugs by administering a test drug to a mammalian cell of claim 3 and observing changes in APP expression and processing or histopathology.
